(19) United States
(12) Patent Application Publication
(10) Pub. No.: US 2012/0231141 A1
(43) Pub. Date: Sep. 13, 2012

(54) BITTERNESS-IMPROVING AGENT FOR BITTER FOOD AND DRINK AND ASTRINGENCY-IMPROVING AGENT FOR ASTRINGENT FOOD AND DRINK

(76) Inventors: Yoshiko Kurobayashi, Kanagawa (JP); Setsuko Nakai, Kanagawa (JP); Kikue Kubota, Tokyo (JP)

(21) Appl. No.: 13/509,424

(22) PCT Filed: Nov. 12, 2010
(86) PCT No.: PCT/JP2010/070168

§ 371 Date: May 11, 2012

(30) Foreign Application Priority Data
Nov. 12, 2009 (JP) 2009-259307
May 18, 2010 (JP) 2010-114365

(51) Int. Cl. C07D 307/88; A23L 1/226

(52) U.S. Cl. 426/536; 549/302; 549/307; 426/442

(57) ABSTRACT

[Object] To improve bitterness or astringency, for example, to reduce stimulative bitterness or astringency of bitter or astringent food and drink, without imparting unnecessary taste or aroma to the food and drink.

[Solution] A bitterness- or astringency-improving agent for bitter or astringent food and drink includes phthalides as an active ingredient. Addition of a very small amount of phthalides that are not sensed as aroma to food and drink improves undesirable bitterness or astringency of various bitter or astringent food and drink products and enhances or provides pleasant bitterness or astringency.

### TECHNICAL FIELD

The present invention relates to a bitterness-improving agent for bitter food and drink and an astringency-Improving agent for astringent food and drink and, more specifically, relates to a bitterness-improving agent and an astringency-improving agent containing phthalides as active ingredients for improving the bitterness of bitter food and drink and the astringency of astringent food and drink to provide preferred bitterness and astringency.

### BACKGROUND ART

As food and drink having bitterness, for example, fruits such as grapefruit, which contains a bitter substance naringin, vegetables such as bitter gourd, roasted extracts such as coffee, flavoring substances, chemicals such as peptides, and protein hydrolysates are known as those having bitterness. In the case of a drink containing grapefruit juice or coffee, bitterness is usually reduced by, for example, adding various types of sweeteners, and in the case of removing bitterness of protein hydrolysates, for example, it has been proposed a method for improving bitterness or astringency of a protein hydrolysate through treatment with an enzyme derived from squid liver (Patent Literature 1).

Incidentally, food and drink products containing astringent components represented by tannin, for example, tea drinks, contain a large amount of polyphenols. The polyphenols have an antioxidant activity and an active oxygen scavenging ability and are therefore paid attention as healthy food and drink. However, if a large amount of polyphenols are contained, the astringency is increased, and the flavor may become unfavorable. Accordingly, there is a demand for bitterness or astringency improvement that can reduce or remove the astringency. As the bitterness- or astringency-improving method for such astringency, for example, a method of adding an extract derived from sugar cane to food and drink has been proposed (Patent Literature 2).

### CITATION LIST

### Patent Literature

Patent Literature 1: Japanese Patent Laid-Open No. Hei 7-115913
Patent Literature 2: Japanese Patent Laid-Open No. 2002-34471

### SUMMARY OF INVENTION

### Technical Problem

As described above, fundamentally, stimulative taste or sensation such as bitterness and astringency is basically not desirably contained in a large amount in food and is desired to be improved.

### Solution to Problem

The present inventors have diligently studied for improvement of bitterness and astringency of food and drink and, as a result, have found that an ingredient derived from Umbelliferae plants is involved in enhancement of, for example, savor and flavor of chicken broth and that the ingredient is phthalides (J. Agric. Food Chem., Vol. 56, No. 2, 512-516, 2008). Accordingly, the present inventors have assumed that phthalides further include various flavor-improving effects and have carried out further studies. As a result, surprisingly, it has been found that mere addition of a very small amount of phthalides that are not sensed as aroma to food and drink improves undesirable bitterness or astringency of various food and drink products and enhances or provides pleasant bitterness or astringency. Thus, the present invention has been accomplished.

Accordingly, the present invention provides the following aspects:

(1) a bitterness-improving agent for bitter food and drink, the agent including phthalides as an active ingredient;
(2) an astringency-improving agent for astringent food and drink, the agent including phthalides as an active ingredient;
(3) the astringency-improving agent according to aspect (2), wherein the astringent food and drink is catechin-containing food;
(4) the bitterness-improving agent according to aspect (1), wherein the improvement of bitterness is masking of the bitterness;
(5) the astringency-improving agent according to aspect (2) or (3), wherein the improvement of astringency is masking of the astringency;
(6) the bitterness-improving agent according to aspect (1) or (4), wherein the phthalides are at least one selected from sedanenolide, sedanolide, 3-n-butylphthalide, and 3-butylidenephthalide;
(7) the astringency-improving agent according to aspect (2), (3), or (5), wherein the phthalides are at least one selected from sedanenolide, sedanolide, 3-n-butylphthalide, and 3-butylidenephthalide;
(8) a bitterness-improving agent composition including the bitterness-improving agent for bitter food and drink according to aspect (1), (4), or (6) in an amount of 10 ppb to 1% as phthalides;
(9) an astringency-improving agent composition including the astringency-improving agent for astringent food and drink according to aspect (2), (3), (5), or (7) in an amount of 10 ppb to 1% as phthalides;
(10) a method of improving bitterness of bitter food and drink, the method including adding the bitterness-improving agent according to aspect (1), (4), or (6) to the bitter food and drink in an amount of 0.01 ppb to 10 ppm as phthalides;
(11) a method of improving astringency of astringent food and drink, the method including adding the astringency-improving agent according to aspect (2), (3), (5), or (7) to the astringent food and drink in an amount of 0.01 ppb to 10 ppm as phthalides;
(12) a method of improving bitterness of bitter food and drink, the method including adding the bitterness-improving agent composition according to aspect (8) to the bitter food and drink in an amount of 0.01 ppb to 10 ppm as phthalides; and
(13) a method of improving astringency of astringent food and drink, the method including adding the astringency-improving agent composition according to aspect (9) to the astringent food and drink in an amount of 0.01 ppb to 10 ppm as phthalides.

### Advantageous Effects of Invention

According to the present invention, it is possible to improve bitterness or astringency of bitter or astringent food and drink by, for example, masking the bitterness or the astringency without imparting unnecessary taste or aroma to the food and drink.

### DESCRIPTION OF EMBODIMENTS

The phthalides used in the present invention refer to a compound with phthalide skeleton among a group of compounds characteristically existing in essential oil of Umbelliferae plants and having characteristic spicy herbal medicine smell. Specifically, the phthalides refer to, for example, sedanenolide, sedanolide, 3-n-butylphthalide, 3-butylidenephthalide, ligustilide, cnidilide, isocnidilide, neocnidilide, methyl sedanoate, and 3-butylhexahydrophthalide. Among these compounds, particularly preferred are sedanenolide, sedanolide, 3-n-butylphthalide, and 3-butylidenephthalide. The phthalides may be synthetic products or may be prepared by obtaining an extract or essential oil from an Umbelliferae plant by, for example, water or solvent extraction or steam distillation and then purifying the extract or essential oil by, for example, various known methods.

These compounds may be used alone or in a combination of two or more thereof. Furthermore, essential oil of, for example, Cnidium rhizome, Angelica sinensis, Lavage, or celery, or an extract, such as oleoresin, containing the above-mentioned compounds may be used directly, or the compounds may be used as an ingredient contained in a flavor preparation.

Incidentally, essential oil and oleoresin of Umbelliferae plants are natural raw materials and are preferred as the bitterness- or astringency-improving agent of the present invention from the viewpoint of safety and relief. However, the essential oil or oleoresin contains, in addition to the phthalides, hydrocarbons such as limonene, myrcene, β-caryophyllene, α-selinene, β-selinene, and γ-selinene and has characteristic greenish aroma. Such greenish aroma is a pleasant savor when the essential oil or oleoresin is used for providing the savor of Umbelliferae plants, as in usual flavoring. However, in the use as the bitterness- or astringency-improving agent of the present invention, these hydrocarbons unnecessarily provide the greenish aroma and are therefore preferable to be reduced or removed. In particular, selinenes have strong greenish aroma specific to Umbelliferae plants and are thus desired to be reduced or removed as much as possible. Among these hydrocarbons, substances having a relatively low boiling point such as limonene (boiling point: 176° C., atmospheric pressure) are easily reduced by distillation (temperature: 50 to 70° C., pressure: 500 to 1000 Pa) using, for example, a usual rectifying column. Selinenes (β-selinene, boiling point: 260° C., atmospheric pressure), which are sesquiterpene hydrocarbons, and phthalides (sedanenolide, boiling point: 367° C., atmospheric pressure) both belong to a group having relatively high boiling points as those of flavoring compounds, and these compounds therefore remain in the still residue in distillation using a usual rectifying column, and it is difficult to separate these compounds. Accordingly, it is necessary to remove selinenes from essential oil or oleoresin of Umbelliferae plants by a simple and low-cost method.

The hydrocarbons including selinenes can be reduced or removed from essential oil or oleoresin of Umbelliferae plants by various types of chromatography, but as a simple and industrially practical method, molecular distillation can be recommended. The molecular distillation is performed under high vacuum conditions and is a purification method by distillation in such a manner that evaporated molecules arrive and are condensed at a cooling surface without causing collision with other molecules. Any type of apparatus or instrument that can be applied to this method can be used without particular limitation, and a batch or continuous molecular distillation apparatus is usually used and is classified based on the system into a falling film molecular distillation apparatus and a centrifugal film molecular distillation apparatus. Among these apparatuses, from the viewpoint of forming stable thin-film conditions, the centrifugal film molecular distillation apparatus, in particular, a continuous-type centrifugal film molecular distillation apparatus is preferably used.

The conditions for removing the hydrocarbons with a centrifugal film molecular distillation apparatus are, for example, a temperature of 90 to 110° C. and a pressure of 10 to 30 Pa. Under such conditions, not only hydrocarbons having low boiling points such as limonene but also selinenes, which are sesquiterpene hydrocarbons, can be distilled away, and phthalides can be obtained as a still residue. In the case of subjecting essential oil to molecular distillation as a raw material, one-time molecular distillation can provide the bitterness- or astringency-improving agent of the present invention as the still residue containing 60% or more of phthalides. Furthermore, in the case of a raw material containing nonvolatile components such as oleoresin, after removal of hydrocarbons under the above-mentioned conditions, the still residue containing phthalides is subjected to molecular distillation again to evaporate phthalides under conditions of a temperature of 140 to 160° C. and a pressure of 10 to 30 Pa to provide the bitterness- or astringency-improving agent of the present invention as the distillate containing 60% or more of phthalides.

Incidentally, the molecular distillation itself is generally used not only for purification of, for example, oils and fats but also for removing the terpene fraction from essential oil of, for example, orange. However, it is not known to apply the molecular distillation for reducing or removing unnecessary components such as limonene and selinenes from essential oil or oleoresin of Umbelliferae plants to purify a bitterness- or astringency-improving agent.

The present invention relates to bitterness- and astringency-improving agents for bitter or astringent food and drink, including phthalides as the active ingredients, and relates to bitterness- and astringency-improving agents that can improve bitterness or astringency of bitter or astringent food and drink by adding these phthalides in the bitter or astringent food and drink in such a low amount that is not sensed as spicy herbal medicine smell specific to the phthalides.

Next, the present invention will be described with reference to individual embodiments below.

In one embodiment, the present invention provides bitter food and drink of which bitterness is masked. Examples of the bitter food and drink include carbonated drinks; juices, fruit juice drinks, and juice-containing soft drinks of citrus fruits (e.g., grapefruit, orange, or lemon); fruit pulp drinks and granules-containing fruit drinks of citrus fruits; vegetables such as tomato, green pepper, celery, cucurbitaceous plant, bitter gourd, carrot, potato, asparagus, bracken fern, and flowering fern and vegetable-based drinks containing these vegetables; vegetable soups; soy milk drinks; coffee drinks; green tea drinks; oolong tea drinks; and drinks containing herbal medicines or herbs.

In the bitter food and drink of the present invention, the blending ratio of the phthalides in the final product is preferably 0.01 ppb to 10 ppm, more preferably 0.1 ppb to 1 ppm. Furthermore, in many cases, these phthalides are practically added to a formulation, and the formulation is added to a final product. In such a case, since the amount of the formulation added to the final product is about 0.01 to 1%, the amount of the phthalides added to the formulation is 10 ppb to 1%, more preferably 100 ppb to 1000 ppm. In this blending ratio, the bitterness is masked, and so the bitter food and drink becomes easy to eat or drink.

In another embodiment of the present invention, the present invention provides an astringent food and drink, in particular, tea drinks, of which astringency is masked. Specific examples of the astringent food and drink include tea leaves such as green tea, black tea, and oolong tea and drinks produced using tea leaf extracts. The contents of polyphenols of tea drinks vary depending on their production methods, and thereby the degree of astringency also varies. However, the astringency can be masked by adding the phthalides in an amount of 0.01 ppb to 10 ppm, more preferably 0.1 ppb to 1 ppm in a final product. Furthermore, in many cases, these phthalides are practically added to a formulation, and the formulation is added to a final product. In such a case, since the amount of the formulation added to the final product is about 0.01 to 1%, the amount of the phthalides added to the formulation is 10 ppb to 1%, more preferably 100 ppb to 1000 ppm. In this blending ratio, the astringency is masked, and so the astringent food and drink becomes easy to eat or drink.

In the bitter or astringent food and drink of the present invention, the blending ratio of the phthalides in the final product is preferably 0.01 ppb to 10 ppm, more preferably 0.1 ppb to 1 ppm. Furthermore, in many cases, these phthalides are practically added to a formulation, and the formulation is added to a final product. In such a case, since the amount of the formulation added to the final product is about 0.01 to 1%, the amount of the phthalides added to the formulation is 10 ppb to 1%, more preferably 100 ppb to 1000 ppm. In this blending ratio, the stimulative sensation of bitterness or astringency of bitter or astringent food and drink is reduced to improve the bitterness or astringency accompanied by the stimulative sensation caused by salt to provide moderate bitterness and astringency.

The concentration of phthalides to be added in the improvement of bitterness or astringency of food and drink is as described above, and even if the concentration of the phthalides is higher than the above-mentioned range, the effect of improving bitterness or astringency is not increased. Accordingly, it is not necessary to add the phthalides in an amount exceeding the above-mentioned range. However, if desired, for example, in the case of imparting the aroma specific to phthalides to food and drink, the phthalides may be added to the food and drink in an amount exceeding the above-mentioned range.

The present invention will be more specifically described by the following examples, but is not limited thereto.

### EXAMPLES

### Example 1

### Masking of Bitterness of Carbonated Water

Sedanenolide was added as a bitterness-improving agent (masking agent for bitterness) of the present invention to sufficiently deaerated pure water at the concentrations shown in the following Table 4, and then carbon dioxide gas was sealed in each bottle at a pressure of 294 KPa to obtain carbonated water of the present invention.

### Comparison of Flavor

Table 1 shows the comparison evaluation of flavor by 10 panelists for the carbonated water containing or not containing the product of the present invention.

TABLE 1
Bitterness evaluation of carbonated water

| Concentration of | | Evaluation | | | Flavor | |
|---|---|---|---|---|---|---|
| sedanenolide | | A | B | N | judgment | Note |
| Sedanenolide-free | | | | | | |
| 1 | ppt | 10 | 0 | 0 | 0 | |
| 0.01 | ppb | 7 | 3 | 0 | 0 | |
| 0.1 | ppb | 5 | 5 | 0 | 0 | |
| 1 | ppb | 4 | 6 | 0 | 0 | |
| 0.01 | ppm | 2 | 2 | 6 | 1 | |
| 0.1 | ppm | 0 | 3 | 7 | 4 | |
| 1 | ppm | 0 | 3 | 7 | 5 | |
| 10 | ppm | 0 | 2 | 8 | 0 | slight spicy aroma |
| | | | | | | was sensed |

Evaluation
A: the number of panelists who felt no difference from sedanenolide-free
B: the number of panelists who felt some reduction in bitterness
N: the number of panelists who hardly felt bitterness
Flavor judgment: the number of panelists who judged it as most proper bitterness

As shown in Table 1, it was judged that though carbonated water not containing sedanenolide had bitterness, the bitterness of carbonated water containing 0.01 ppb to 10 ppm of sedanenolide was masked. When the amount of sedanenolide was about 0.1 ppb to 1 ppm, particularly good results were obtained. In an amount of 10 ppm, though bitterness itself was masked, a slight spicy aroma other than bitterness was also sensed. Even in a low concentration of 0.01 ppb, some panelists felt some reduction in bitterness. Thus, it was revealed that the effect of masking bitterness can be obtained even at a low concentration.

### Example 2

### Masking of Bitterness of Grapefruit Juice

3-n-Butylphthalide was added as a bitterness-improving agent (masking agent for bitterness) of the present invention to commercially available grapefruit juice at the concentrations shown in the following Table 2 to obtain grapefruit juice.

### Comparison of Flavor

Table 2 shows the comparison evaluation of flavor by 10 panelists for the grapefruit juice containing or not containing the product of the present invention.

TABLE 2
Bitterness evaluation of grapefruit juice

| Concentration of | | Evaluation | | | Flavor | |
|---|---|---|---|---|---|---|
| 3-n-butylphthalide | | A | B | N | judgment | Note |
| 3-n-Butylphthalide-free | | | | | | |
| 1 | ppt | 9 | 1 | 0 | 0 | |
| 0.01 | ppb | 7 | 2 | 1 | 0 | |
| 0.1 | ppb | 4 | 6 | 0 | 0 | |
| 1 | ppb | 3 | 7 | 0 | 0 | |
| 0.01 | ppm | 2 | 6 | 2 | 2 | |
| 0.1 | ppm | 0 | 4 | 6 | 5 | |
| 1 | ppm | 0 | 4 | 6 | 3 | |
| 10 | ppm | 0 | 3 | 7 | 0 | slight spicy aroma |
| | | | | | | was sensed |

Evaluation
A: the number of panelists who felt no difference from 3-n-butylphthalide-free
B: the number of panelists who felt some reduction in bitterness
N: the number of panelists who hardly felt bitterness
Flavor judgment: the number of panelists who judged it as most proper bitterness

As shown in Table 2, it was judged that though grapefruit juice not containing 3-n-butylphthalide had bitterness, the bitterness of grapefruit juice containing 0.01 ppb to 10 ppm of 3-n-butylphthalide was masked. When the amount of 3-n-butylphthalide was about 0.1 ppb to 1 ppm, particularly good results were obtained. In an amount of 10 ppm, though bitterness itself was masked, a slight spicy aroma other than bitterness was also sensed. Even in a low concentration of 0.01 ppb, some panelists felt some reduction in bitterness. Thus, it was revealed that the effect of masking bitterness can be obtained even at a low concentration.

### Example 3

### Masking of Bitterness of Green Tea Drink

Five thousand grams of hot water (containing 0.03% of sodium ascorbate) of 60° C. was added to 100 g of Chinese green tea (second picking tea) produced by Josei (heat treatment by steaming), followed by extraction for 5 min while sometimes stirring. Subsequently, solid-liquid separation with flannel filter was performed to obtain 4500 g of an extract (Bx 0.6°). To the extract, 0.04 g of tannase (Kikkoman Corporation: 5000 U/g) was added. The resulting mixture was left to stand at 40° C. for 1 hour to decompose the gallate-type catechin to non-gallate-type catechin (gallate-type catechin has strong astringency, and non-gallate-type catechin has strong bitterness), followed by heating at 90° C. for 1 min to inactivate the enzyme. After cooling to 20° C., the extract was adjusted to Bx 0.5° with deionized water to obtain green tea extract having strong bitterness. Sedanolide was added as a taste-improving agent (masking agent for bitterness) of the present invention to the resulting extract at the concentrations shown in the following Table 3 to obtain green tea drinks.

### Comparison of Flavor

Table 3 shows the comparison evaluation of flavor by 10 panelists for the green tea drinks containing or not containing the product of the present invention.

TABLE 3
Bitterness evaluation of green tea drink

| Concentration of | | Evaluation | | | Flavor | |
|---|---|---|---|---|---|---|
| sedanolide | | A | B | N | judgment | Note |
| Sedanolide-free | | | | | | |
| 1 | ppt | 10 | 0 | 0 | 0 | |
| 0.01 | ppb | 8 | 2 | 0 | 0 | |
| 0.1 | ppb | 4 | 5 | 1 | 0 | |
| 1 | ppb | 3 | 6 | 1 | 0 | |
| 0.01 | ppm | 2 | 6 | 2 | 2 | |
| 0.1 | ppm | 0 | 5 | 5 | 5 | |
| 1 | ppm | 0 | 5 | 5 | 3 | |
| 10 | ppm | 0 | 5 | 5 | 0 | slight spicy aroma |
| | | | | | | was sensed |

Evaluation
A: the number of panelists who felt no difference from sedanolide-free
B: the number of panelists who fell some reduction in bitterness
N: the number of panelists who hardly felt bitterness
Flavor judgment: the number of panelists who judged it as most proper bitterness

As shown in Table 3, it was judged that though green tea drink not containing sedanolide had bitterness, the bitterness of green tea drink containing 0.01 ppb to 10 ppm of sedanolide was masked. When the amount of sedanolide was about 0.1 ppb to 1 ppm, particularly good results were obtained. In an amount of 10 ppm, though bitterness itself was masked, a slight spicy aroma other than bitterness was also sensed. Even in a low concentration of 0.01 ppb, some panelists felt some reduction in bitterness. Thus, it was revealed that the effect of masking bitterness can be obtained even at a low concentration.

### Example 4

### Masking of Astringency of Green Tea Drink

Five thousand grams of hot water (containing 0.03% of sodium ascorbate) of 95° C. was added to 100 g of domestic green tea (second picking tea), followed by extraction for 5 min while sometimes stirring. Subsequently, solid-liquid separation with flannel filter was performed to obtain 4500 g of an extract (Bx 0.7°). This extract was adjusted to Bx 0.5° with deionized water to obtain green tea extract having strong astringency. Sedanenolide was added as a taste-improving agent (masking agent for astringency) of the present invention to the resulting extract at the concentrations shown in the following Table 4 to obtain green tea drinks.

### Comparison of Flavor

Table 4 shows the comparison evaluation of flavor by 10 panelists for the green tea drinks containing or not containing the product of the present invention.

TABLE 4
Astringency evaluation of green tea drink

| Concentration of | | Evaluation | | | Flavor | |
|---|---|---|---|---|---|---|
| sedanenolide | | A | B | N | judgment | Note |
| Sedanenolide-free | | | | | | |
| 1 | ppt | 10 | 0 | 0 | 0 | |
| 0.01 | ppb | 8 | 2 | 0 | 0 | |
| 0.1 | ppb | 6 | 3 | 1 | 0 | |
| 1 | ppb | 5 | 5 | 0 | 0 | |
| 0.01 | ppm | 3 | 6 | 1 | 2 | |
| 0.1 | ppm | 0 | 3 | 7 | 5 | |
| 1 | ppm | 0 | 3 | 7 | 3 | |
| 10 | ppm | 0 | 2 | 8 | 0 | slight spicy aroma |
| | | | | | | was sensed |

Evaluation
A: the number of panelists who felt no difference from sedanenolide-free
B: the number of panelists who felt some reduction in astringency
N: the number of panelists who hardly felt astringency
Flavor judgment: the number of panelists who judged it as most proper astringency

As shown in Table 4, it was judged that though green tea drink not containing sedanenolide had astringency, the astringency of green tea drink containing 0.01 ppb to 10 ppm of sedanenolide was masked. When the amount of sedanenolide was about 0.1 ppb to 1 ppm, particularly good results were obtained. In an amount of 10 ppm, though bitterness itself was masked, a slight spicy aroma other than bitterness was also sensed. Even in a low concentration of 0.01 ppb, some panelists felt some reduction in bitterness. Thus, it was revealed that the effect of masking bitterness can be obtained even at a low concentration.

### Reference Example 1

### Purification of Essential Oil by Precision Distillation

As a commercially available celery seed essential oil (Reference Product 1), an essential oil having a component composition composed of 1.86% of 3-n-butylphthalide, 5.4% of sedanenolide, 0.32% of sedanolide, 66.2% of limonene, 12.9% of β-selinene, and 13.3% of others (measured by gas chromatography) was used. Reference Product 1 (120.5 g) was subjected to reduced pressure precision distillation under the following conditions. The pressure was gradually reduced from 1000 Pa to 500 Pa at an internal temperature of 54° C., and when distraction of distillate stopped, the precision distillation was terminated to obtain a distillate and a still residue (Reference Product 2). The compositions of the resulting distillate and still residue were as follows: 80.3 g of the distillate portion was composed of 1.43% of 3-n-butylphthalide, 3.72% of sedanenolide, 0.27% of sedanolide, 85.5% of limonene, 3.5% of β-selinene, and 5.58% of others, and 40.8 g of the still residue portion (Reference Product 2) was composed of 7.07% of 3-n-butylphthalide, 20.5% of sedanenolide, 1.22% of sedanolide, 0% of limonene, 43.4% of β-selinene, and 27.81% of others.

As described above, in the precision distillation, all of limonene was evaporated, and 85% or more of the distillate portion was limonene. On the other hand, though the still residue portion contained concentrated phthalides, it was recognized that β-selinene was also concentrated in the still residue portion.

### Example 5

### Purification of Oleoresin by Molecular Distillation

As a commercially available celery oleoresin (Reference Product 3), oleoresin having volatile component composition composed of 5.0% of 3-n-butylphthalide, 41.3% of sedanenolide, 2.5% of sedanolide, 30.4% of limonene, 8.2% of β-selinene, and 12.6% of others was used.

To Reference Product 3 (362.6 g), 72.9 g (20% of Reference Product 3) of rice salad oil was added. The resulting mixture was sufficiently mixed and fed to a centrifugal film molecular distillation apparatus. Thin film distillation was performed by allowing the mixture to flow into a heat transfer surface from a feed nozzle at a rate of 3.6 g/min under a reduced pressure of 15 to 28 Pa, allowing cooling water to flow in a condenser, and cooling a cold trap with dry ice/acetone. On this occasion, the treatment liquid was heated under conditions of a temperature of 100° C. for about 1.0 sec for a thin film having a thickness of 0.1 mm on average. After distillation, 25.1 g of a cold trap portion, 10.6 g of a cooling water trap portion, and 399.8 g of a still residue portion were obtained.

The volatile component composition of the still residue was 6.9% of 3-n-butylphthalide, 58.3% of sedanenolide, 3.0% of sedanolide, 8.3% of limonene, 13.2% of β-selinene, and 10.3% of others).

The still residue portion (399.8 g) was fed to the centrifugal film molecular distillation apparatus again, and thin film distillation was performed by allowing the still residue to flow into the heat transfer surface from the feed nozzle at a rate of 3.6 g/min under a reduced pressure of 15 to 28 Pa, allowing cooling water to flow in the condenser, and cooling the cold trap with dry ice/acetone. On this occasion, the treatment liquid was heated under conditions of a temperature of 150° C. for about 1.0 sec for a thin film having a thickness of 0.1 mm on average. Only the distillation portion after distillation was used as Invention Product 1 (yield: 54.7 g).

The component composition of Invention Product 1 was 8.0% of 3-n-butylphthalide, 65.5% of sedanenolide, 3.5% of sedanolide, 0.6% of limonene, 10.9% of β-selinene, and 11.5% of others. The still residue portion (345.1 g) of the second molecular distillation hardly contained volatile components that are detected by gas chromatography.

### Example 6

### Preparation of Bitterness- or Astringency-Masking Powder

An oil phase portion was prepared by mixing and dissolving 19.6 g of medium chain fatty acid triglyceride and 30 g of SAIB in 0.093 g of 3-n-butylphthalide, 0.27 g of sedanenolide, and 0.016 g of sedanolide. Separately, 955 g of Pinedex No. 2 and 50 g of sucrose fatty acid ester having an HLB of 15 were added and dissolved in 680 g of soft water, and the resulting solution was heat-sterilized at 85° C. for 15 min. This solution was cooled to about 40° C., and 50 g of the oil phase portion prepared above was then poured thereto while stirring with a TK-homomixer (manufactured by Tokusyu Kika Kogyo Co., Ltd.). The resulting mixture was further stirred at 5000 rpm for 5 min for emulsification to obtain 1690 g of emulsion. This emulsion was dried using a spray dryer (manufactured by Niro A/S, Mobile Minor) at a blast temperature of 150° C. and at an exhaust temperature of 80° C. to obtain 900 g of bitterness and/or astringency masking powder (Invention Product 2: containing 0.0093% of 3-n-butylphthalide, 0.027% of sedanenolide, and 0.0016% of sedanolide).

1. A bitterness-improving agent for bitter food and drink, the agent comprising phthalides as an active ingredient.

2. An astringency-improving agent for astringent food and drink, the agent comprising phthalides as an active ingredient.

3. The astringency-improving agent according to claim 2, wherein the astringent food and drink is catechin-containing food.

4. The bitterness-improving agent according to claim 1, wherein the improvement of bitterness is masking of the bitterness.

5. The astringency-improving agent according to claim 2, wherein the improvement of astringency is masking of the astringency.

6. The bitterness-improving agent according to claim 1, wherein the phthalides are at least one selected from sedanenolide, sedanolide, 3-n-butylphthalide, and 3-butylidenephthalide.

7. The astringency-improving agent according to claim 2, wherein the phthalides are at least one selected from sedanenolide, sedanolide, 3-n-butylphthalide, and 3-butylidenephthalide.

8. A bitterness-improving agent composition comprising the bitterness-improving agent for bitter food and drink according to claim 1 in an amount of 10 ppb to 1% as phthalides.

9. An astringency-improving agent composition comprising the astringency-improving agent for astringent food and drink according to claim 2 in an amount of 10 ppb to 1% as phthalides.

10. A method of improving bitterness of bitter food and drink, the method comprising adding the bitterness-improving agent according to claim 1 to the bitter food and drink in an amount of 0.01 ppb to 10 ppm as phthalides.

11. A method of improving astringency of astringent food and drink, the method comprising adding the astringency-improving agent according to claim 2 to the astringent food and drink in an amount of 0.01 ppb to 10 ppm as phthalides.

12. A method of improving bitterness of bitter food and drink, the method comprising adding the bitterness-improving agent composition according to claim 8 to the bitter food and drink in an amount of 0.01 ppb to 10 ppm as phthalides.

13. A method of improving astringency of astringent food and drink, the method comprising adding the astringency-improving agent composition according to claim 9 to the astringent food and drink in an amount of 0.01 ppb to 10 ppm as phthalides.
